(19) United States
(12) Patent Application Publication
(10) Pub. No.: US 2024/0299630 A1
(43) Pub. Date: Sep. 12, 2024

(54) Coated Medical Devices and Methods of Coating

(71) Applicant: Bard Access Systems, Inc., Salt Lake City, UT (US)

(72) Inventors: Ejaz Sajedul Haque, Salt Lake City, UT (US); Nicholas P. Papadopoulos, Salt Lake City, UT (US); Brendan Laine, Salt Lake City, UT (US); James L. Freasier, Salt Lake City, UT (US)

(21) Appl. No.: 18/120,330

(22) Filed: Mar. 10, 2023

(51) Int. Cl. A61L 29/08; A61L 29/16

(52) U.S. Cl. CPC A61L 29/085; A61L 29/16; A61L 2300/404; A61L 2300/442; A61L 2300/608; A61L 2420/02

(57) ABSTRACT

Coated medical devices include coated catheters. For example, a coated catheter can include a catheter tube of a tubular substrate and a coating thereover. The tubular substrate can be of a first polymeric material transparent to electromagnetic radiation in a range of visible light. The coating can be of a second polymeric material anchored to the tubular substrate by chain ends of the second polymeric material impregnated in the first polymeric material by way of a spent visible-light photoinitiator. Methods of coating can include methods of coating medical devices such as the coated catheter. For example, a method of coating can include irradiating a tubular substrate impregnated with a visible-light photoinitiator with the foregoing electromagnetic radiation while the tubular substrate is disposed in an aqueous solution of a monomer, thereby initiating a radical polymerization of the monomer and coating the tubular substrate with the coating of the second polymer material.

### BACKGROUND

Light-initiated free-radical polymerization for coating medical devices such as catheters makes use of ultraviolet (“UV”)-light photoinitiators having relatively high absorptivity in a range of UV light such as UVC (e.g., 100-280 nm), UVB (e.g., 280-315 nm), UVC (e.g., 315-400 nm), or a narrower range thereof. UV-light photoinitiators for such free-radical polymerization includes, for example, tert-butyl peroxybenzoate and benzophenone, which have relatively strong UVC absorption peaks at 232 nm and 254 nm, respectively. While the foregoing UV-light photoinitiators can be effective in generating and transferring free radicals for chain-growth polymerization when the UV-light photoinitiators are irradiated in the range of UV light in which they absorb, the UV light, itself, can degrade materials, thereby limiting substrate choices for coated medical devices. In addition, the UV light poses occupational risks, thereby requiring additional safety measures to limit exposure of workers to the UV light.

Disclosed herein are coated medical devices and methods of coating that make use of visible-light photoinitiators.

### SUMMARY

Disclosed herein is a coated catheter including, in some embodiments, a catheter tube including a tubular substrate and a coating over the tubular substrate. The tubular substrate is of a first polymeric material transparent to electromagnetic radiation in a range of visible light. The coating is of a second polymeric material anchored to the tubular substrate by chain ends of the second polymeric material impregnated in the first polymeric material. At least a portion of the chain ends including a spent visible-light photoinitiator.

In some embodiments, the first polymeric material is a thermoplastic polyurethane transparent to electromagnetic radiation in the range of visible light from 400 nm to 650 nm.

In some embodiments, the thermoplastic polyurethane includes a hard segment having one or more sulfur-based chain extenders.

In some embodiments, the thermoplastic polyurethane includes a soft segment having a polycarbonate moiety.

In some embodiments, the thermoplastic polyurethane includes a soft segment having a polyether moiety.

In some embodiments, the spent visible-light photoinitiator is spent camphorquinone or a spent analog of camphorquinone.

In some embodiments, at least another portion of the chain ends of the first polymeric material include a spent coinitiator.

In some embodiments, the spent coinitiator is a spent tertiary amine selected from ethyl-4-dimethylaminobenzoate; 4-(dimethylamino) benzonitrile; and 2-(N,N-dimethylamino)ethyl methacrylate.

In some embodiments, the coating of the second polymeric material is over either an abluminal surface or a luminal surface of the tubular substrate.

In some embodiments, the coating of the second polymeric material is over both an abluminal surface and a luminal surface of the tubular substrate.

In some embodiments, the second polymeric material is a polyacrylate salt or ester.

In some embodiments, the second polymeric material is the polyacrylate salt. At least a portion of functionalized sites of the second polymeric material are functionalized with anionic carboxylate and a cationic therapeutic agent as a counterion.

In some embodiments, the therapeutic agent is an antimicrobial agent.

In some embodiments, the therapeutic agent is chlorhexidine.

In some embodiments, at least another portion of the functionalized sites of the second polymeric material are functionalized with anionic carboxylate and a cationic dye as a counterion. The dye provides a visible indication the coating of the second polymeric material is over the tubular substrate.

In some embodiments, the dye doubles as an antifungal agent.

In some embodiments, the dye is ethyl violet.

In some embodiments, the coated catheter further includes a catheter hub and one or more extension legs. The catheter tube includes a proximal end portion disposed in the catheter hub. Each extension leg of the one-or-more extension legs includes a distal end portion disposed in the catheter hub.

Also disclosed herein is a method of manufacturing a coated catheter. The method includes, in some embodiments, steps or operations for providing a coated tubular substrate. As such, the method includes obtaining an impregnated tubular substrate of a first polymeric material transparent to electromagnetic radiation in a range of visible light. The first polymeric material is impregnated with a visible-light photoinitiator. The method also includes disposing the impregnated tubular substrate in an aqueous solution including a monomer dissolved in the aqueous solution. The method also includes irradiating the impregnated tubular substrate with electromagnetic radiation in the range of visible light to which the first polymeric material is transparent. The photoinitiator initiates a radical polymerization of the monomer upon irradiation of the photoinitiator. The radical polymerization coats the impregnated tubular substrate with a coating of a second polymer material to provide the coated tubular substrate.

In some embodiments, the method further includes disposing a non-impregnated tubular substrate in an organic-solvent solution including the photoinitiator dissolved in the organic-solvent solution. The non-impregnated tubular substrate swells in the organic-solvent solution such that the photoinitiator diffuses into the first polymeric material, thereby impregnating the first polymeric material with the photoinitiator to provide the impregnated tubular substrate in a solvent-swollen form thereof.

In some embodiments, the method further includes disposing the solvent-swollen form of the impregnated tubular substrate in water. Organic solvent diffuses from the first polymeric material into the water, thereby shrinking the solvent-swollen form of the impregnated tubular substrate and trapping the photoinitiator in the first polymeric material.

In some embodiments, the first polymeric material is a thermoplastic polyurethane transparent to electromagnetic radiation in the range of visible light from 400 nm to 650 nm. The thermoplastic polyurethane includes a hard segment having one or more sulfur-based chain extenders and a soft segment having a polycarbonate moiety.

In some embodiments, the visible-light photoinitiator is camphorquinone or an analog of camphorquinone characterized by its absorption of electromagnetic radiation in the range of visible light from 400 nm to 650 nm.

In some embodiments, the impregnated tubular substrate is further impregnated with a coinitiator, the coinitiator being a tertiary amine selected from ethyl-4-dimethylaminobenzoate; 4-(dimethylamino) benzonitrile; and 2-(N,N-dimethylamino)ethyl methacrylate.

In some embodiments, the coating of the second polymeric material is over either an abluminal surface or a luminal surface of the coated tubular substrate.

In some embodiments, the coating of the second polymeric material is over both an abluminal surface and a luminal surface of the coated tubular substrate.

In some embodiments, the second polymeric material is a polyacrylate salt or ester.

In some embodiments, the method further includes steps or operations for providing additional functionality to the coating of the second polymer material. As such, the method also includes disposing the coated tubular substrate in another aqueous solution including a therapeutic agent. A proton or metal cation is thereby exchanged for a cationic therapeutic agent as a counterion to anionic carboxylate in at least a portion of functionalized sites of the second polymeric material.

In some embodiments, the therapeutic agent is an antimicrobial agent.

In some embodiments, the therapeutic agent is chlorhexidine.

In some embodiments, the other aqueous solution further includes a dye. A proton or metal cation is thereby exchanged for a cationic dye as a counterion to anionic carboxylate in at least another portion of functionalized sites of the second polymeric material.

In some embodiments, the dye doubles as an antifungal agent.

In some embodiments, the dye is ethyl violet.

In some embodiments, the method further includes steps or operations for assembling the coated catheter. As such, the method also includes inserting a proximal end portion of the coated tubular substrate into a catheter hub. The coated tubular substrate corresponds to a catheter tube of the coated catheter. The method also includes inserting a distal end portion of an extension leg into the catheter hub for each extension leg of one or more extension legs of the coated catheter.

These and other features of the concepts provided herein will become more apparent to those of skill in the art in view of the accompanying drawings and following description, which describe particular embodiments of such concepts in greater detail.

### DRAWINGS

FIG. 1 illustrates a coated catheter in accordance with some embodiments.

FIG. 2 illustrates a transverse cross section of a catheter tube of the coated catheter in accordance with some embodiments.

FIG. 3 illustrates a transverse cross section of another catheter tube of the coated catheter in accordance with some embodiments.

FIG. 4 illustrates a transverse cross section of yet another catheter tube of the coated catheter in accordance with some embodiments.

FIG. 5 illustrates a transverse cross section of yet another catheter tube of the coated catheter in accordance with some embodiments.

FIG. 6 provides a schematic of the catheter tube of FIG. 3 with a coating anchored to the catheter tube, an antimicrobial agent ionically bonded to the coating, and a dye ionically bonded to the coating in accordance with some embodiments.

FIG. 7 illustrates a genus of transparent thermoplastic polyurethanes for the catheter tube in accordance with some embodiments.

FIG. 8 illustrates another genus of transparent thermoplastic polyurethanes for the catheter tube in accordance with some embodiments.

FIG. 9 provides a schematic of the catheter tube of FIG. 4 illustrating the coating anchored to the catheter tube by spent visible-light photoinitiator and spent coinitiator in accordance with some embodiments.

FIG. 10 provides a schematic illustrating a method of coating the catheter tube in accordance with some embodiments.

FIG. 11 provides a schematic illustrating a mechanism for anchoring the coating to the catheter tube by the spent visible-light photoinitiator and spent coinitiator in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

Unless defined otherwise, all technical and scientific terms used herein have the same meaning as commonly understood by those of ordinary skill in the art.

Again, light-initiated free-radical polymerization for coating medical devices such as catheters makes use of UV-light photoinitiators having relatively high absorptivity in a range of UV light such as UVC (e.g., 100-280 nm), UVB (e.g., 280-315 nm), UVC (e.g., 315-400 nm), or a narrower range thereof. UV-light photoinitiators for such free-radical polymerization includes, for example, tert-butyl peroxybenzoate and benzophenone, which have relatively strong UVC absorption peaks at 232 nm and 254 nm, respectively. While the foregoing UV-light photoinitiators can be effective in generating and transferring free radicals for chain-growth polymerization when the UV-light photoinitiators are irradiated in the range of UV light in which they absorb, the UV light, itself, can degrade materials, thereby limiting substrate choices for coated medical devices. In addition, the UV light poses occupational risks, thereby requiring additional safety measures to limit exposure of workers to the UV light.

Disclosed herein are coated medical devices and methods of coating that make use of visible-light photoinitiators, thereby at least expanding substrate choices for the coated medical devices and reducing the occupational risks associated with using higher-energy UV light. Coated medical devices include, without limitation, coated catheters such as peripherally inserted central catheters (‘PICCs”). For example, a coated catheter can include a catheter tube of a tubular substrate and a coating thereover. The tubular substrate can be of a first polymeric material transparent to electromagnetic radiation in a range of visible light. The coating can be of a second polymeric material anchored to the tubular substrate by chain ends of the second polymeric material impregnated in the first polymeric material by way of a spent visible-light photoinitiator. Methods of coating can include, without limitation, methods of coating medical devices such as the foregoing coated catheter. For example, a method of coating can include irradiating a tubular substrate impregnated with a visible-light photoinitiator with the foregoing electromagnetic radiation while the tubular substrate is disposed in an aqueous solution of a monomer, thereby initiating a radical polymerization of the monomer and coating the tubular substrate with the coating of the second polymer material.

### Medical Devices

Coated medical devices encompass any medical device having a coating like that described herein on an intracorporeal portion of the medical device or an entirety thereof, wherein the intracorporeal portion or entirety of the medical device is configured to reside within a patient for a period of time such as during treatment of the patient. For example, the coated medical devices can include coated catheters, which, in turn, can include coated peripheral (intra)venous catheters (“PIVCs”) and coated central venous catheters (“CVCs”). Coated CVCs, in turn, can include coated ports, coated percutaneous CVCs configured for insertion through skin into a jugular or subclavian vein, coated rapidly insertable central catheters (“RICCs”), coated PICCs configured for insertion through skin into a vein of an arm, and subcutaneous or tunneled CVCs having a coating like that described herein.

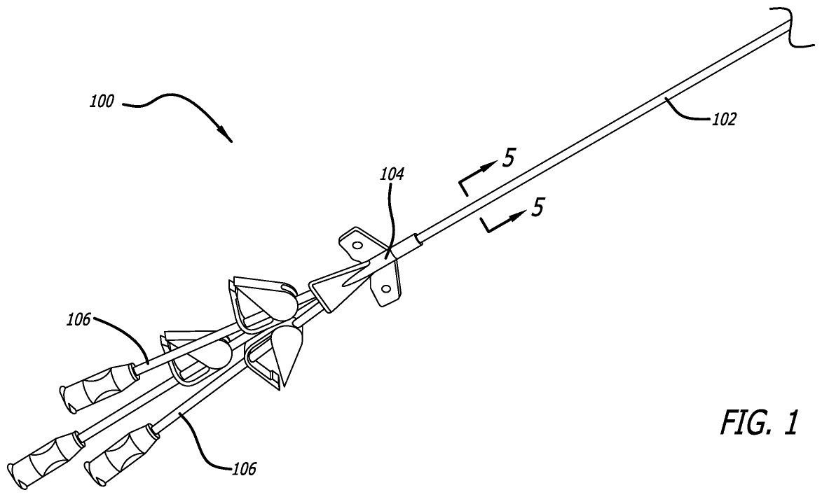

FIG. 1 illustrates a coated catheter 100 in accordance with some embodiments. More specifically, FIG. 1 illustrates a coated PICC in accordance with some embodiments. However, it should be understood that description for the coated catheter 100 is not limited to PICCs but extends to the other coated catheters set forth above as well as other coated medical devices. For example, description of the coating 110 on the catheter tube 102 set forth below extends to a coating on a catheter tube of another coated catheter such as any other coated catheter set forth above. And being that the catheter tube 102 of the coated catheter 100 is the intracorporeal portion thereof, the description of the coating 110 on the catheter tube 102 [[###]] set forth below further extends to a coating on the intracorporeal portion of another coated medical device.

The coated catheter 100 includes a catheter tube 102, a catheter hub 104, and one or more extension legs 106 operably connected in the foregoing order. Indeed, the catheter tube 102 includes a proximal end portion disposed in the catheter hub 104, and each extension leg of the one-or-more extension legs 106 includes a distal end portion disposed in the catheter hub 104.

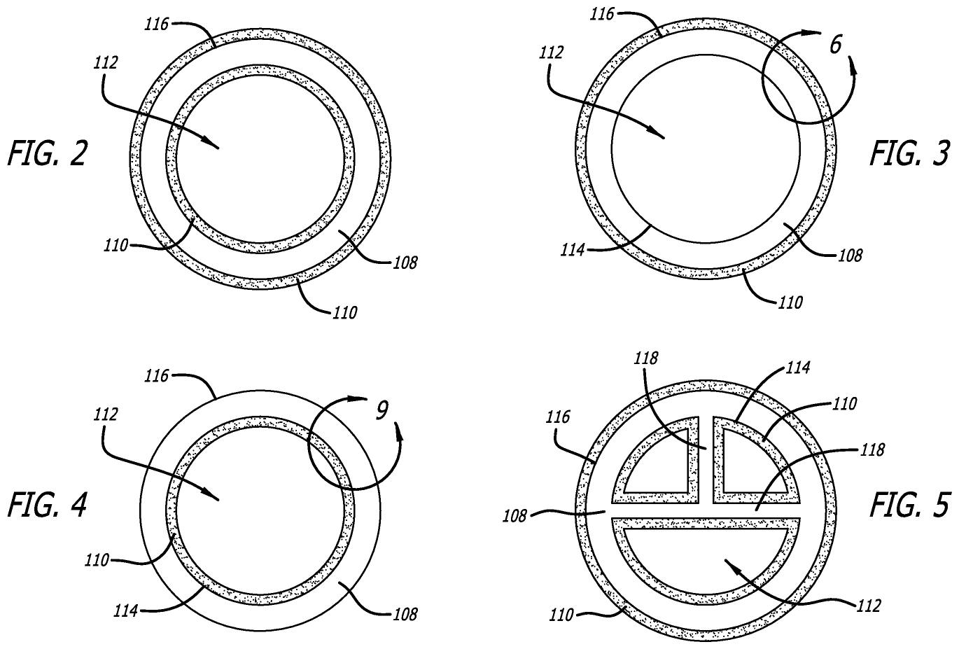

FIGS. 2-5 illustrate transverse cross sections of the catheter tube 102 of the coated catheter 100 in accordance with some embodiments. As shown, the catheter tube 102 includes a tubular substrate 108 and a coating 110 over the tubular substrate 108, which tubular substrate 108 corresponds to the coated tubular substrate 132 [[###]] set forth below in at least some embodiments. The tubular substrate 108 includes at least one lumen 112 therethrough, the foregoing lumen 112 thereby defining at least one luminal surface 114 of the tubular substrate 108 as well as an abluminal surface 116 of the tubular substrate 108. Additional lumens of the tubular substrate 108 are defined by one or more longitudinal septa 118 dividing the foregoing lumen 112 as shown in FIG. 5. Each additional lumen of the additional lumens includes a corresponding luminal surface. The coating 110 can be over any one or more surfaces selected from the abluminal surface 116 and any luminal surface of the tubular substrate 108. In an example, the coating 110 can be over the abluminal surface 116 and each luminal surface of the tubular substrate 108 as shown in FIGS. 2 and 5. In another example, the coating 110 can be over the abluminal surface 116 of the tubular substrate 108 as shown in FIG. 3. In yet another example, the coating 110 can be over the luminal surface 114 of the tubular substrate 108 as shown in FIG. 4.

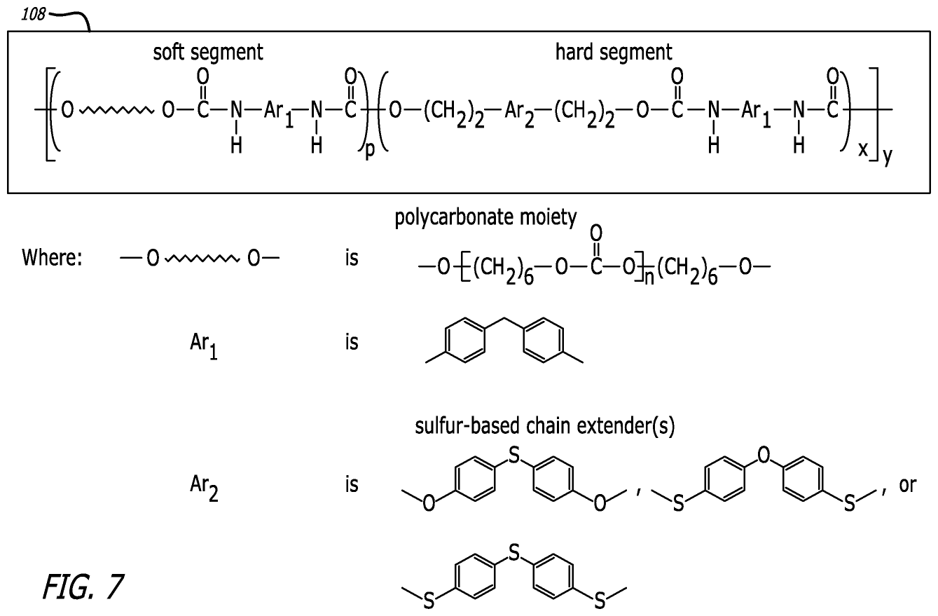

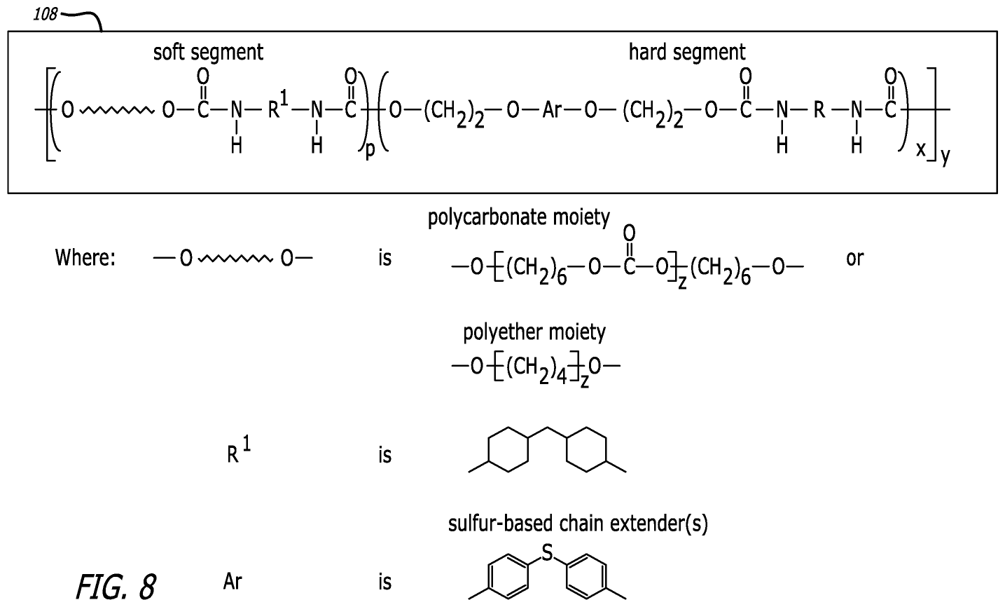

The tubular substrate 108 is of a first polymeric material transparent to electromagnetic radiation in a range of visible light. For example, the first polymeric material can be a thermoplastic polyurethane transparent to electromagnetic radiation in the range of visible light from 400 nm to 650 nm, including 400 nm to 550 nm, such as 450 nm to 500 nm, for example, 460 nm to 470 nm. As shown in FIGS. 7 and 8, such a thermoplastic polyurethane can include a hard segment having one or more sulfur-based chain extenders and a soft segment having a polycarbonate or a polyether moiety. Some of the thermoplastic polyurethanes encompassed by FIGS. 7 and 8 can be found in the following references along with their preparation, each of which references is incorporated herein it its entirety: 1) Rogulska et al. Chem. Pap. 2017, 71, 1195-1204; and Rogulska et al. Polym. Bull. 2018, 75, 1211-1235. However, it should be understood the first polymeric material is not limited to the thermoplastic polyurethanes of FIGS. 7 and 8 or even thermoplastic polyurethanes. Indeed, the first polymeric material can be any medically suitable polymeric material that is transparent to electromagnetic radiation in the foregoing range(s) of visible light and stable to conditions of the method set forth below. Notably, the tubular substrate 108 of the first polymeric material can include one or more dyes or pigments integrated therein so long as the tubular substrate 108 remains transparent to electromagnetic radiation in the foregoing range(s) of visible light.

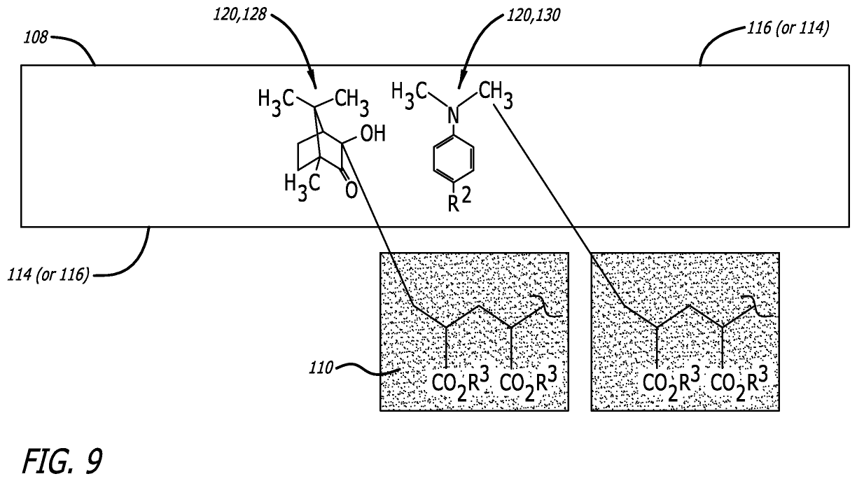

The coating 110 is of a second polymeric material anchored to the tubular substrate 108 by chain ends 120 of the second polymeric material impregnated in the first polymeric material, which advantageously make the coating 110 of the second polymeric material resistant to delamination. The second polymeric material can be a polyacrylic acid (—R³ is —H in FIG. 9), polyacrylate salt, polyacrylate ester, or combination thereof, wherein functionalized sites 122 of the second polymeric material are functionalized with a combination of functional groups selected from carboxy, carboxylate salt, and carboxylate ester in the foregoing combination. The acrylic acid or acrylate residue of the second polymeric material is either unsubstituted or substituted with an alkyl, cycloalkyl, aryl, or heteroaryl group. When present, the carboxylate salt includes one or more counterions selected from a metal cation, cationic therapeutic agent 124, and cationic dye 126. Further, when present, the carboxylate ester includes an organyl group (e.g., —R³ in FIG. 9) selected from an alkyl, cycloalkyl, aryl, or heteroaryl group.

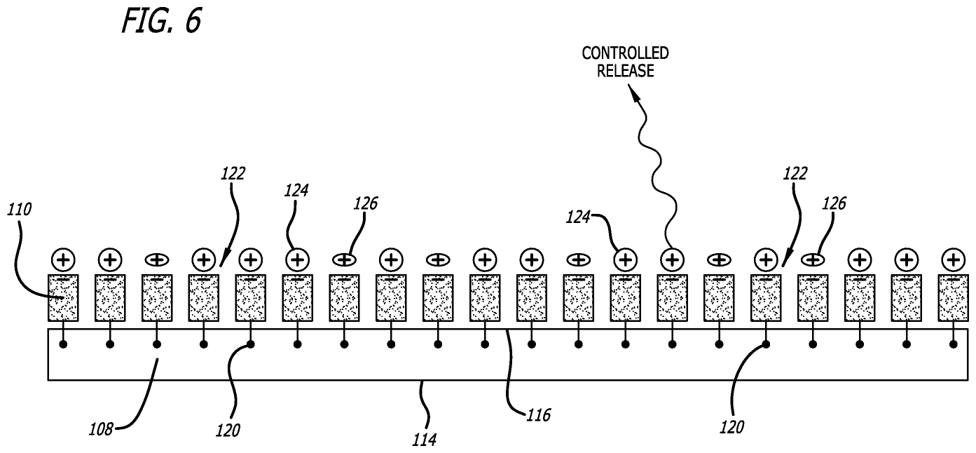

Advantageously, when the second polymeric material includes the polyacrylate salt, at least a portion of the functionalized sites 122 of the second polymeric material functionalized with anionic carboxylate can include one of the cationic therapeutic agent 124 or the cationic dye 126 as the counterion, for example, the cationic therapeutic agent 124. Further, at least another portion of the functionalized sites 122 of the second polymeric material can include the other one of the cationic therapeutic agent 124 or the cationic dye 126 as the counterion, for example, the cationic dye 126. Such an example is shown in the schematic of FIG. 6, wherein at least a portion of the functionalized sites 122 of the second polymeric material include the cationic therapeutic agent 124 as the counterion and at least another portion of the functionalized sites 122 of the second polymeric material include the cationic dye 126 as the counterion, the cationic therapeutic agent 124 and the cationic dye 126 thereby ionically bonded to the coating 110 or the second polymeric material thereof. The therapeutic agent 124 can include, but is not limited to, an antimicrobial agent such as an antimicrobial small molecule, for example, chlorhexidine, or an antimicrobial peptides for example, dalbavancin, daptomycin, oritavancin, teicoplanin, telavancin, or vancomycin, which antimicrobial agent can be slowly or controllably released from the coating 110 or the second polymeric material thereof to prevent bacterial colonization, thereby addressing catheter-related bloodstream infections (“CRBSIs”). The dye 126 can include, but is not limited to, a triarylmethane dye, for example, a methyl violet dye (e.g., methyl violet 10B) or ethyl violet. The dye 126 functions to provide a visible indication the coating 110 of the second polymeric material is present over the tubular substrate 108, but the dye 126 can also function as a therapeutic agent in some embodiments. For example, each of dye 126 of methyl violet 10B and ethyl violet can double as an antimicrobial or antifungal agent, which can enhance any antimicrobial activity of the cationic therapeutic agent 124 with additional antimicrobial activity or complement any antimicrobial activity of the cationic therapeutic agent 124 with antifungal activity when both the cationic therapeutic agent 124 and the cationic dye 126 are ionically bonded to the coating 110 or the second polymeric material thereof.

As to the second polymeric material being anchored to the tubular substrate 108 by the chain ends 120 of the second polymeric material impregnated in the first polymeric material, it should be understood the chain ends 120 are primarily initiation ends of polymer chains of the second polymeric material as opposed to termination ends of the polymer chains. That is, the chain ends 120 of the second polymeric material impregnated in the first polymeric material are those from which the polymer chains of the second polymeric material propagated. Being that the tubular substrate 108 is impregnated with the photoinitiator 136 prior to initiating the radical polymerization set forth in the method below, at least a portion of the chain ends 120 of the second polymeric material impregnated in the first polymeric material include a spent visible-light photoinitiator 128 from which the polymer chains of the second polymeric material propagated. Such a spent visible-light photoinitiator 128 can include spent camphorquinone as shown in FIG. 9 or a spent analog of camphorquinone such as spent carboxylated camphorquinone. Further, being that the tubular substrate 108 can be impregnated with the coinitiator 142 prior to initiating the radical polymerization set forth in the method below, at least another portion of the chain ends 120 of the second polymeric material impregnated in the first polymeric material can include a spent coinitiator 130 from which the polymer chains of the second polymeric material propagated. Such a spent coinitiator 130 can include a spent tertiary amine selected from spent ethyl-4-dimethylaminobenzoate, 4-(dimethylamino) benzonitrile, and 2-(N,N-dimethylamino)ethyl methacrylate. Indeed, FIG. 9 shows spent ethyl-4-dimethylaminobenzoate when —R² is —CO₂CH₂CH₃ and spent 4-(dimethylamino) benzonitrile when —R² is —CN. Even further, should the tubular substrate 108 be impregnated with a polymerization accelerator prior to initiating the radical polymerization set forth in the method below, at least another portion of the chain ends 120 of the second polymeric material impregnated in the first polymeric material can include a spent polymerization accelerator. Such a spent polymerization accelerator can include spent diphenyliodonium chloride, which can be indicated by the chain ends 120 of the second polymeric material including phenyl groups.

### Methods

Methods include methods of manufacturing coated medical devices, which methods, in turn, include methods of coating medical devices or pieces thereof in the manufacturing of the coated medical devices. For example, the methods of manufacturing coated medical devices include a method of manufacturing a coated catheter such as the coated catheter 100, which method, in turn, includes a method of coating a catheter or a piece thereof in the manufacturing of the coated catheter 100. However, like that set forth above it should be understood that description for the method of manufacturing the coated catheter 100, again, a PICC, is not limited to manufacturing PICCs but extends to manufacturing the other coated catheters set forth above as well as manufacturing the other coated medical devices. For example, description of coating the catheter tube 102 set forth below extends to coating a catheter tube of another coated catheter such as any other coated catheter set forth above. And being that the catheter tube 102 of the coated catheter 100 is the intracorporeal portion thereof, the description of coating the catheter tube 102 set forth below further extends to coating the intracorporeal portion of another coated medical device.

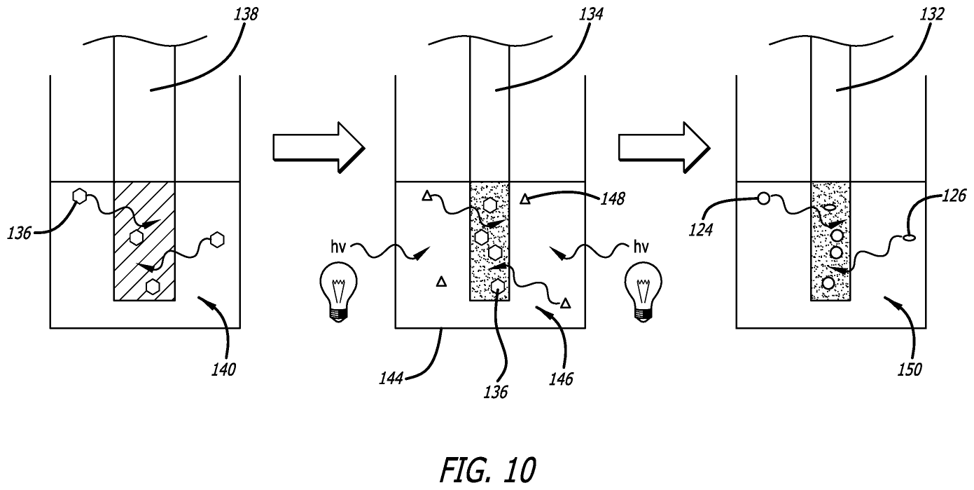

The method of manufacturing the coated catheter 100 includes, like that set forth above, a method of coating a piece of the coated catheter 100 such as the catheter tube 102, which is followed by a method of assembling the coated catheter 100 with the foregoing catheter tube 102. As set forth below, such a method of manufacturing the coated catheter 100 includes various steps or operations beginning with the steps or operations for providing a coated tubular substrate 132 as shown in FIG. 10, which steps or operations can include an impregnating operation, a coating operation, and a functionalizing operation. Such a method of manufacturing the coated catheter 100 includes various steps or operations continuing with the steps or operations for assembling the coated catheter 100 with the foregoing coated tubular substrate 132 as the catheter tube 102, optionally after cutting the coated tubular substrate 132 to size and tipping it with a catheter tip.

As to the impregnating operation, it can include obtaining an impregnated tubular substrate 134 of the first polymeric material impregnated with at least a visible-light photoinitiator 136. Obtaining the impregnated tubular substrate 134 includes disposing a non-impregnated tubular substrate 138 of the first polymeric material in an organic-solvent solution 140 including the photoinitiator 136 dissolved in the organic-solvent solution 140 by a relatively polar organic solvent or mixture of organic solvents, including a mixture of protic or aprotic organic solvents, such as a mixture of protic and aprotic organic solvents, for example, a mixture of 2-propanol and 2-butanone. Upon disposing the non-impregnated tubular substrate 138 in the organic-solvent solution 140, the non-impregnated tubular substrate 138 swells in the organic-solvent solution 140, thereby allowing the photoinitiator 136 to diffuse into the first polymeric material, which, in turn, impregnates the first polymeric material with the photoinitiator 136 to provide the impregnated tubular substrate 134 in a solvent-swollen form thereof. Notably, any lumen of the non-impregnated tubular substrate 138 can be stoppered or otherwise closed off before disposing the non-impregnated tubular substrate 138 in the organic-solvent solution 140 to prevent the photoinitiator 136 from entering the lumen and diffusing into the first polymeric material of the corresponding luminal surface, thereby providing control over which luminal surfaces of the coated tubular substrate 132 are coated. Subsequently disposing the solvent-swollen form of the impregnated tubular substrate 134 in water allows the organic solvent to diffuse from the first polymeric material into the water, thereby shrinking the solvent-swollen form of the impregnated tubular substrate 134 and trapping the photoinitiator 136 in the first polymeric material to provide the impregnated tubular substrate 134 of the first polymeric material impregnated with at least the photoinitiator 136.

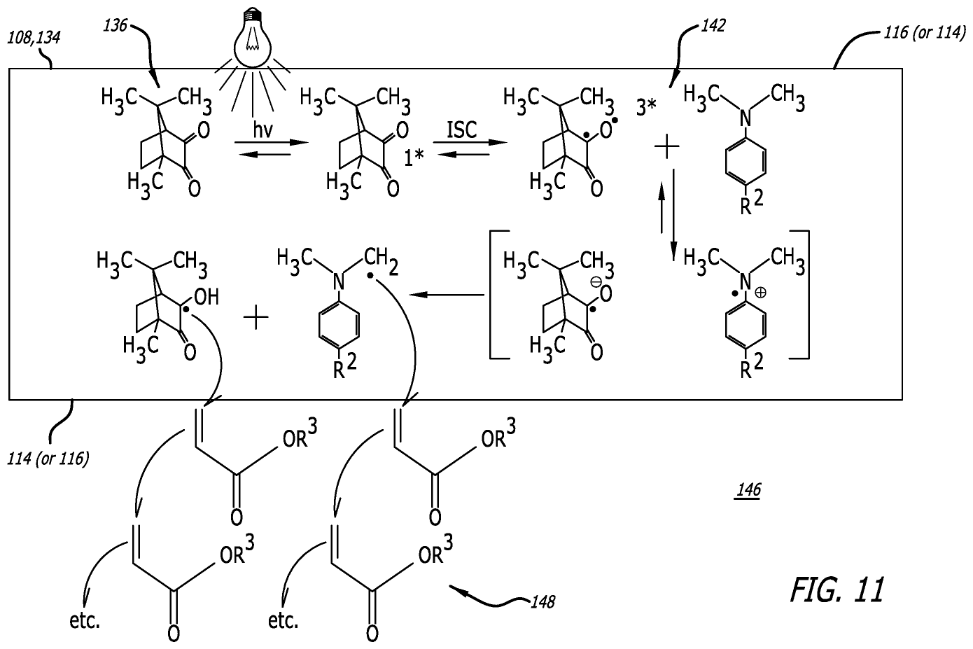

The photoinitiator 136 can be characterized by its absorption of electromagnetic radiation in the range of visible light from 400 nm to 650 nm, including 400 nm to 550 nm, such as 450 nm to 500 nm, for example, 460 nm to 470 nm. Such a photoinitiator 136 can include, but is not limited to, camphorquinone as shown in FIG. 11 or an analog thereof such as carboxylated camphorquinone. (See Kamoun et al. Arab. J. Chem. 2016, 9(5), 745-754. Because initiation of radical polymerization with visible-light photoinitiators such as camphorquinone can benefit from coinitiators, the organic-solvent solution 140 can further include a coinitiator 142 dissolved in the organic-solvent solution 140 along with the photoinitiator 136, which coinitiator 142 likewise impregnates the first polymeric material as set forth above when the coinitiator 142 is present in the organic-solvent solution 140. Such a coinitiator 142 can include, but is not limited to, a tertiary amine selected from ethyl-4-dimethylaminobenzoate, 4-(dimethylamino) benzonitrile, and 2-(N,N-dimethylamino)ethyl methacrylate. Indeed, FIG. 11 shows ethyl-4-dimethylaminobenzoate when —R² is —CO₂CH₂CH₃ and 4-(dimethylamino) benzonitrile when —R² is —CN. Lastly, because radical polymerization with visible-light photoinitiators such as camphorquinone can be accelerated with polymerization accelerators, the organic-solvent solution 140 can further include a polymerization accelerator dissolved in the organic-solvent solution 140 along with the photoinitiator 136, which polymerization accelerator likewise impregnates the first polymeric material as set forth above when the polymerization accelerator is present in the organic-solvent solution 140. Such a polymerization accelerator can include, but is not limited to, diphenyliodonium chloride.

As to the coating operation, it can include disposing the impregnated tubular substrate 134 in a suitable reactor 144 (e.g., a quartz reactor) including an aqueous solution 146 having a monomer 148 dissolved in the aqueous solution 146 and irradiating the impregnated tubular substrate 134 with one or more lamps or light-emitting diodes (“LEDs”) 150, thereby providing electromagnetic radiation in the range of visible light to which the first polymeric material is transparent and the photoinitiator 136 absorbs. The photoinitiator 136, which remains trapped in the impregnated tubular substrate 134 in accordance with the swell-preventing aqueous solution 146 of the monomer 148, initiates a radical polymerization of the monomer 148 from the impregnated tubular substrate 134 or wall thereof including the photoinitiator 136 upon irradiation of the photoinitiator 136. As shown in FIG. 11 by way of camphorquinone as the photoinitiator 136 and ethyl-4-dimethylaminobenzoate (when —R² is —CO₂CH₂CH₃) or 4-(dimethylamino) benzonitrile (when —R² is —CN) as the coinitiator 142, upon absorption of the electromagnetic radiation in the range of visible light to which the photoinitiator 136 absorbs (e.g., 468 nm for camphorquinone), an excited singlet state of the photoinitiator 136 is formed, and, through intersystem crossing, a triplet state of the photoinitiator 136 is subsequently formed, thereby providing a diradical of the photoinitiator 136. Electron transfer between the diradical of the photoinitiator 136 and the coinitiator 142 results in an exciplex between a radical anion of the photoinitiator 136 and a radical cation of the coinitiator 142. Further, by way of proton transfer within the exciplex, a ketyl radical of the photoinitiator 136 is formed along with an alkyl radical of the coinitiator 142, both of which are capable of propagating their radicals in the radical polymerization of the monomer 148. Indeed, as shown in FIG. 11, each radical of the ketyl and alkyl radicals can react with the monomer 148 in the aqueous solution 146 in which the impregnated tubular substrate 134 is disposed to coat the impregnated tubular substrate 134 with the second polymer material, thereby providing the coated tubular substrate 132 with the second polymer material anchored to the coated tubular substrate 132.

As set forth above, the second polymeric material anchored to the coated tubular substrate 132 can be a polyacrylic acid, polyacrylate salt, polyacrylate ester, or combination thereof. As such, the monomer 148 dissolved in the aqueous solution 146 into which the impregnated tubular substrate 134 is disposed can be an acrylic acid (—R³ is —H in FIG. 11) or acrylate ester. The acrylic acid or acrylate ester can be either unsubstituted or substituted with an alkyl, cycloalkyl, aryl, or heteroaryl group. Further, when the acrylate ester is present, the organyl group (e.g., —R³ in FIG. 11) of the acrylate ester is selected from an alkyl, cycloalkyl, aryl, or heteroaryl group.

As to the functionalizing operation, it can include modifying the functionalized sites 122 of the second polymer material such as those functionalized with anionic carboxylate to include the one-or-more counterions set forth above, the one-or-more counterions selected from the metal cation, cationic therapeutic agent 124, and cationic dye 126. Accordingly, the functionalizing operation can include disposing the coated tubular substrate 132 in another aqueous solution 150 including the metal cation, cationic therapeutic agent 124, cationic dye 126, or a combination thereof. In an example, the functionalizing operation can include disposing the coated tubular substrate 132 in the other aqueous solution 150 including the cationic therapeutic agent 124, thereby exchanging a proton or metal cation for the cationic therapeutic agent 124 as the counterion to anionic carboxylate in at least a portion of the functionalized sites 122 of the second polymeric material. In another example, the functionalizing operation can include disposing the coated tubular substrate 132 in the other aqueous solution 150 including the cationic dye 126, thereby exchanging a proton or metal cation for the cationic dye 126 as the counterion to anionic carboxylate in at least a portion of the functionalized sites 122 of the second polymeric material. In yet another example, the functionalizing operation can include disposing the coated tubular substrate 132 in the other aqueous solution 150 including both the cationic therapeutic agent 124 and the cationic dye 126, thereby exchanging a proton or metal cation for the cationic therapeutic agent 124 as the counterion to anionic carboxylate in at least a portion of the functionalized sites 122 of the second polymeric material as well as exchanging a proton or metal cation for the cationic dye 126 as the counterion to anionic carboxylate in another portion of the functionalized sites 122 of the second polymeric material.

As to an assembling operation, it can include inserting a proximal end portion of the coated tubular substrate 132 into the catheter hub 104, optionally after cutting the coated tubular substrate 132 to size and tipping it, as set forth above. Such a coated tubular substrate 132 thereby corresponds to the catheter tube 102 of the coated catheter 100. Further, the assembling operation can include inserting a distal end portion of an extension leg into the catheter hub 104 for each extension leg of the one-or-more extension legs 106 of the coated catheter 100.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A coated catheter, comprising:
a catheter tube including:
a tubular substrate of a first polymeric material transparent to electromagnetic radiation in a range of visible light; and
a coating of a second polymeric material over the tubular substrate, the coating anchored to the tubular substrate by chain ends of the second polymeric material impregnated in the first polymeric material, at least a portion of the chain ends including a spent visible-light photoinitiator.

2. The coated catheter of claim 1, wherein the first polymeric material is a thermoplastic polyurethane transparent to electromagnetic radiation in the range of visible light from 400 nm to 650 nm.

3. The coated catheter of claim 2, wherein the thermoplastic polyurethane includes a hard segment having one or more sulfur-based chain extenders.

4. The coated catheter of claim 2, wherein the thermoplastic polyurethane includes a soft segment having a polycarbonate moiety.

5. The coated catheter of claim 2, wherein the thermoplastic polyurethane includes a soft segment having a polyether moiety.

6. The coated catheter of claim 1, wherein the spent visible-light photoinitiator is spent camphorquinone or a spent analog of camphorquinone.

7. The coated catheter of claim 1, wherein at least another portion of the chain ends of the first polymeric material include a spent coinitiator.

8. The coated catheter of claim 7, wherein the spent coinitiator is a spent tertiary amine selected from ethyl-4-dimethylaminobenzoate; 4-(dimethylamino) benzonitrile; and 2-(N,N-dimethylamino)ethyl methacrylate.

9. The coated catheter of claim 1, wherein the coating of the second polymeric material is over either an abluminal surface or a luminal surface of the tubular substrate.

10. The coated catheter of claim 1, wherein the coating of the second polymeric material is over both an abluminal surface and a luminal surface of the tubular substrate.

11. The coated catheter of claim 1, wherein the second polymeric material is a polyacrylate salt or ester.

12. The coated catheter of claim 11, wherein the second polymeric material is the polyacrylate salt, at least a portion of functionalized sites of the second polymeric material being functionalized with anionic carboxylate and a cationic therapeutic agent as a counterion.

13. The coated catheter of claim 12, wherein the therapeutic agent is an antimicrobial agent.

14. The coated catheter of claim 12, wherein the therapeutic agent is chlorhexidine.

15. The coated catheter of claim 12, wherein at least another portion of the functionalized sites of the second polymeric material are functionalized with anionic carboxylate and a cationic dye as a counterion, thereby providing a visible indication the coating of the second polymeric material is over the tubular substrate.

16. The coated catheter of claim 15, wherein the dye doubles as an antifungal agent.

17. The coated catheter of claim 15, wherein the dye is ethyl violet.

18. The coated catheter of claim 1, further comprising:
a catheter hub, the catheter tube including a proximal end portion disposed in the catheter hub; and
one or more extension legs, each extension leg of the one-or-more extension legs including a distal end portion disposed in the catheter hub.

19. A method of manufacturing a coated catheter, comprising:
obtaining an impregnated tubular substrate of a first polymeric material transparent to electromagnetic radiation in a range of visible light, the first polymeric material impregnated with a visible-light photoinitiator;
disposing the impregnated tubular substrate in an aqueous solution including a monomer dissolved in the aqueous solution; and
irradiating the impregnated tubular substrate with electromagnetic radiation in the range of visible light to which the first polymeric material is transparent, the photoinitiator initiating a radical polymerization of the monomer upon irradiation of the photoinitiator, thereby coating the impregnated tubular substrate with a coating of a second polymer material to provide a coated tubular substrate.

20. The method of claim 19, further comprising:
disposing a non-impregnated tubular substrate in an organic-solvent solution including the photoinitiator dissolved in the organic-solvent solution, the non-impregnated tubular substrate swelling in the organic-solvent solution such that the photoinitiator diffuses into the first polymeric material, thereby impregnating the first polymeric material with the photoinitiator to provide the impregnated tubular substrate in a solvent-swollen form thereof.

21. The method of claim 20, further comprising:
disposing the solvent-swollen form of the impregnated tubular substrate in water, organic solvent diffusing from the first polymeric material into the water, thereby shrinking the solvent-swollen form of the impregnated tubular substrate and trapping the photoinitiator in the first polymeric material.

22. The method of claim 19, wherein the first polymeric material is a thermoplastic polyurethane transparent to electromagnetic radiation in the range of visible light from 400 nm to 650 nm, the thermoplastic polyurethane including a hard segment having one or more sulfur-based chain extenders and a soft segment having a polycarbonate moiety.

23. The method of claim 22, wherein the visible-light photoinitiator is camphorquinone or an analog of camphorquinone characterized by its absorption of electromagnetic radiation in the range of visible light from 400 nm to 650 nm.

24. The method of claim 19, wherein the impregnated tubular substrate is further impregnated with a coinitiator, the coinitiator being a tertiary amine selected from ethyl-4-dimethylaminobenzoate; 4-(dimethylamino) benzonitrile; and 2-(N,N-dimethylamino)ethyl methacrylate.

25. The method of claim 19, wherein the coating of the second polymeric material is over either an abluminal surface or a luminal surface of the coated tubular substrate.

26. The method of claim 19, wherein the coating of the second polymeric material is over both an abluminal surface and a luminal surface of the coated tubular substrate.

27. The method of claim 19, wherein the second polymeric material is a polyacrylate salt or ester.

28. The method of claim 27, further comprising:
disposing the coated tubular substrate in another aqueous solution including a therapeutic agent, thereby exchanging a proton or metal cation for a cationic therapeutic agent as a counterion to anionic carboxylate in at least a portion of functionalized sites of the second polymeric material.

29. The method of claim 28, wherein the therapeutic agent is an antimicrobial agent.

30. The method of claim 28, wherein the therapeutic agent is chlorhexidine.

31. The method of claim 28, wherein the other aqueous solution further includes a dye, thereby exchanging a proton or metal cation for a cationic dye as a counterion to anionic carboxylate in at least another portion of functionalized sites of the second polymeric material.

32. The method of claim 31, wherein the dye doubles as an antifungal agent.

33. The method of claim 31, wherein the dye is ethyl violet.

34. The method of claim 19, further comprising:
inserting a proximal end portion of the coated tubular substrate into a catheter hub, the coated tubular substrate corresponding to a catheter tube of the coated catheter, and
inserting a distal end portion of an extension leg into the catheter hub for each extension leg of one or more extension legs of the coated catheter.
